(19) United States
(12) Patent Application Publication
(10) Pub. No.: US 2022/0031709 A1
(43) Pub. Date: Feb. 3, 2022

(54) INHIBITION OF MICROSOMAL PROSTAGLANDIN E2 (PGE2) SYNTHASE-1 (MPGES-1)

(71) Applicant: University of Kentucky Research Foundation, Lexington, TX (US)

(72) Inventors: Chang-Guo Zhan, Lexington, KY (US); Fang Zheng, Lexington, KY (US); Shuo Zhou, Lexington, KY (US); Ziyuan Zhou, Lexington, KY (US)

(21) Appl. No.: 17/393,210

(22) Filed: Aug. 3, 2021

Related U.S. Application Data
(60) Provisional application No. 63/060,386, filed on Aug. 3, 2020.

(51) Int. Cl. A61K 31/546; A61K 31/427; A61P 29/00; A61P 31/14; A61P 19/02; A61P 9/14

(52) U.S. Cl. CPC A61K 31/546; A61K 31/427; A61P 9/14; A61P 31/14; A61P 19/02; A61P 29/00

(57) ABSTRACT

Disclosed methods of inhibiting microsomal prostaglandin E2 (PGE₂) synthase-1 (mPGES-1) make use of ceftriaxone, aztreonam, cefotetan, pharmaceutically-acceptable salts thereof, and combinations thereof.

### RELATED APPLICATIONS

This application claims priority from U.S. Provisional Application Ser. No. 63/060,386 filed Aug. 3, 2020, the entire disclosure of which is incorporated herein by this reference.

### TECHNICAL FIELD

The presently-disclosed subject matter generally relates to inhibition of microsomal prostaglandin E2 (PGE₂) synthase-1 (mPGES-1). In particular, certain embodiments of the presently-disclosed subject matter relate to the use of ceftriaxone, aztreonam, cefotetan, and pharmaceutically-acceptable salts thereof as mPGES-1 inhibitors.

### INTRODUCTION

Coronavirus disease 2019 (COVID-19), caused by severe acute respiratory syndrome coronavirus 2 (SARS-CoV-2) (Rodriguez-Morales et al., 2020; Zhou et al., 2020a) has been characterized by an overexuberant inflammatory response (Stebbing et al., 2020) or a hyperinflammation.(Mehta et al., 2020) Due to the overexuberant inflammatory response, COVID-19 patients could experience sudden deterioration of the disease in about one or two weeks after onset (Zhang et al., 2020), causing pneumonia, called COVID-19 pneumonia (Chua et al., 2020), and acute respiratory distress syndrome (ARDS)—the leading cause of mortality.(Mehta et al., 2020)

SARS-CoV-2 enters target cells via its spike protein binding to angiotensin-converting enzyme 2 (ACE2). ACE2 has a relatively high expression in respiratory epithelial cells, particularly nasal goblet and ciliated cells within human airways.(Sungnak et al., 2020) After viral invasion, various cellular responses including viral cell death mechanism and (early and later) inflammatory processes may occur, depending on the type of cells targeted.(Smeitink et al., 2020)

Particularly, the infected nasal goblet and ciliated cells will undergo cell death via apoptosis, necrosis, and pyroptosis, releasing pathogen- and damage-associated molecular patterns to activate the innate immune response; the activation involves recruitment of granulocytes to the injured tissue and release of inflammatory mediators including proinflammatory cytokines and lipid mediators such as prostaglandins (PGs) to evoke an acute inflammatory process (hours to days) to clear the pathogens and damaged tissues.(Smeitink et al., 2020)

The infected mucosal mast cells in the nasal cavity and submucosal respiratory tract will cause an early inflammatory response. As a result, SARS-CoV-2 invasion and the responses of the different cells of the respiratory tract will lead to progressive therapy resistant inflammation.(Smeitink et al., 2020) Potential antiviral drugs, such as hydroxychloroquine and others (Kupferschmidt and Cohen, 2020; Wang et al., 2020b) tested so far show conflicting results, although remdesivir may help hospitalized COVID-10 patients recover faster.(NPR, 2020) It is desirable to develop new therapeutic strategies.

Generally speaking, coronaviruses are large, lipid-enveloped, positive-sense, single-stranded RNA viruses. High levels of prostaglandin E2 (PGE₂) were detected in the cells infected by this type of viruses compared to the uninfected cells.(Sander et al., 2017)

It has been proposed that prostaglandin E₂ (PGE₂), known as the principal proinflammatory prostaglandin (Ding et al., 2018a), is an important factor contributing to COVID-19 hyperinflammatory (or cytokine storm) and immune responses.(Smeitink et al., 2020). Particularly, it is known that alveolar macrophages participate in the activation of innate and adaptive host immune response in response to the respiratory infection, and that the activation will be inhibited by the increased PGE₂ level, produced by an inducible enzyme known as microsomal PGE₂ synthase-1 (mPGES-1).

Prostaglandin E2 (PGE₂) is the central biomarker for not only COVID-19 hyperinflammation, but also many other inflammation-related diseases such as all types of acute and chronic pain, arthritis, stroke, sepsis, pneumonia, airway inflammation, heart failure, typhoid fever, and vascular inflammation such as abdominal aortic aneurysms (AAAs).(Gomez et al., 2013). In all these inflammation-related disease conditions, PGE₂ concentrations are much higher than those in healthy individuals. For example, it was reported that the concentrations of PGE₂ in urine samples of COVID-19 patients (mean: 170 ng/ml) were significantly higher than those of PGE₂ in urine samples of healthy individuals (mean: 18.8 ng/ml), and that the measured PGE₂ concentrations in the urine samples positively correlate with the progression of COVID-19.(Hong et al., 2020).

The well-known pre-existing severity factors of COVID-19, including gender, ageing, and obesity, are consistent with the previously observed differences in PGE₂ levels associated with these factors. Particularly, higher PGE₂ levels were observed in aged animals and obese individuals, and the pre-existing increased PGE₂ levels might cause higher sensitivity to COVID-19 (Smeitink et al. 2020), which may explain the more severe disease state in older and/or obese patients with COVID-19. It was also reported that freshly isolated and immediately lipopolysaccharide (LPS)-stimulated human neutrophils from males produced more PGE₂ than cells from females (Pace et al., 2020), which may explain the more severe disease state in males following COVID-19 infection.

It was also reported that freshly isolated and immediately lipopolysaccharide (LPS)-stimulated human neutrophils from males produced more PGE₂ than cells from females (Pace et al., 2020), which may also explain the more severe disease state in males following COVID-19 infection. In addition, thrombosis was observed in the severe COVID-19 patients, which is also consistent with the previous report that PGE₂ exacerbates arterial thrombosis and atherothrombosis through platelet EP3 receptors.(Gross et al., 2007)

It is also known that PGE₂ is involved in both the inflammation and immunity pathways, and the PGE₂ over-production can lead to a cytokine storm which causes a variety of adverse effects (Aliabadi et al., 2020), and the proposed molecular mechanism (Aliabadi et al., 2020) by which PGE₂ production increases in response to COVID-19 disease is consistent with the observed direct correlation between the PGE₂ levels and the severity of COVID-19 disease.(Hong et al., 2020).

In general, tissue damage induces overproduction and release of PGE₂ which is known as the principal proinflammatory agent.(Hanaka et al., 2009; Koeberle and Werz, 2015; Radmark and Samuelsson, 2010; Serhan and Levy, 2003) The PGE₂ levels correlate with various proinflammatory cytokines and, hence, overproduction of PGE₂ upregulates various proinflammatory cytokines, such as interleukin (IL)-6, IL-8, and tumor necrosis factor-α (TNF-α). (Schoenberger et al., 2012; St-Jacques and Ma, 2011).

It should also be noted that proinflammatory cytokines, such as interleukin (IL)-6, IL-10, and tumor necrosis factor-α (TNF-α) (Blanco-Melo et al., 2020, Kox et al., 2020), were also over-expressed in patients of COVID-19 and many other inflammation-related diseases. For example, elevated TNF-α and IL-6 levels were detected in the patients of COVID-19 (n=46), sepsis with ARDS (n=51), sepsis without ARDS (n=15), out-of-hospital cardiac arrest (OHCA; n=30), and multiple trauma (n=62).(Kox et al., 2020). The TNF-α and IL-6 levels in the sepsis patients (with or without ARDS) are even significantly higher than the corresponding concentrations in the COVID-19 patients.(Kox et al., 2020).

Notably, the PGE₂ levels correlate with the proinflammatory cytokines and, hence, overproduction of PGE₂ upregulates various proinflammatory cytokines, such as TNF-α and IL-6.(Schoenberger et al., 2012; St.-Jacques et al., 2011). In addition, Janus Kinase (JAK) was also proposed as a potential target for an anti-inflammatory treatment (JAK inhibitor) of COVID-19 in combination with an antiviral treatment.(Stebbling et al., 2020; Stebbing et al. 2020b). JAK is a family of intracellular signaling molecules associated with cytokines. It has been demonstrated that JAK activation participates in PGE₂-induced inflammatory hyperalgesia and, thus, a JAK inhibitor (AG490) was able to block this in vitro effect of PGE₂.(Vieira et al., 2016).

As such, it would be useful to effectively suppress PGE₂ overproduction under the overexuberant inflammatory conditions associated with COVID-19. In addition, other inflammation-related diseases, such as stroke (Li et al., 2020), sepsis (Kox et al., 2020), pneumonia (Bormann et al., 2020), airway inflammation (asthma) (Insuela et al., 2020), heart failure (Reis et al., 2020), typhoid fever (Kaithwas et al., 2011), rheumatoid arthritis (Apostolova et al., 2020), various forms of pain (Vieira et al., 2016; Ma et al., 2019), aging (Minhas et al., 2021), vascular inflammation (Gomez et al., 2013), and many other neurological disorders (Yimer et al., 2019), and those infected by other viruses in the same family with SARS-CoV-2 are also related to elevated PGE₂ levels.

PGE₂ biosynthesis (Kudo and Murakami, 2005) starts from arachidonic acid (AA), a polyunsaturated fatty acid present in phospholipids. The AA is first converted to prostaglandin H2 (PGH₂) by cyclooxygenase (COX)-1 and COX-2.(Kudo and Murakami, 2005) Then, PGH₂ is converted to PGE₂ by mPGES-1, an inducible enzyme, which is induced strongly in inflammatory state while cytosolic PGES (cPGES, a keeper enzyme which provides basal level of PGE₂ for physiological homeostasis (Tanioka et al., 2000)) remains unchanged. Thus, mPGES-1 is mainly responsible for the overproduction of PGE₂ without affecting the basal PGE₂ production.

In comparison, currently available, traditional nonsteroidal anti-inflammatory drugs (NSAIDs), which inhibit COX-1/2, block PGH₂ production. Hence, NSAIDs block the syntheses of all physiologically required prostaglandins (PGs), including PGI₂, PGD₂, PGF_{2α}, and TXA₂, in addition to PGE₂, downstream of PGH₂. (Cheng et al., 2006b). This is why sufficiently effective inhibition of COX-1/2 by traditional NSAIDs are usually associated with serious adverse effects.

Notably, the first generation of NSAIDs, such as aspirin and ibuprofen, weakly and non-selectively inhibit both COX-1 and COX-2. For example, ibuprofen has IC₅₀=13 μM against COX-1 and IC₅₀=370 μM against COX-2. Understandably, the low inhibitory activity is associated with the low efficacy in the treatment of inflammation and pain. As a second generation of NSAIDs, celecoxib (Celebrex), rofecoxib (Vioxx), and valdecoxib (Bextra) can potently and selectively inhibit COX-2. However, the COX-2-selective inhibitors still have a number of serious adverse side effects, as they still increase the risk of fatal heart attack or stroke and cause stomach or intestinal bleeding etc. Due to the serious adverse side effects, rofecoxib and valdecoxib were withdrawn from the market although celecoxib still remains in clinical use with a limited dosage form.

Compared to the traditional NSAIDs, a selective mPGES-1 inhibitor should be much safer, because the mPGES-1 inhibition does not affect the production of basal PGE₂ and other PGs; mPGES-1 as a promising drug target has been confirmed by various knockout studies. (Cheng et al., 2006a; Engblom et al., 2003; Kojima et al., 2008; Kojima et al., 2011; Saha et al., 2005; Sampey et al., 2005; Trebino et al., 2003; Wang et al., 2008; Wang et al., 2006). Indeed, the recently reported drug design and discovery study (Ding et al., 2018a) demonstrated that a potent and selective mPGES-1 inhibitor at an even extremely high oral dose (up to 5 g/kg) did not cause any toxic signs in mice during our observation for 14 days. In comparison, only 50 mg/kg celecoxib administered orally was very toxic for stomach and other issues of mice, and bleeding ulcer was observed at gastric mucosa.(Ding et al., 2018a).

Hence, blocking the PGE₂ over-production, such as by mPGES-1 inhibition, not only can directly block/attenuate the hyperinflammatory response, but also may enhance host immune response against viral infection. (Smeitink et al., 2020). For these reasons, lowering the upregulated PGE₂ levels, such as by using selective inhibition of mPGES-1, without inhibiting biosynthesis of other PGs, is a promising therapeutic strategy to treat the patients affected by the coronavirus family of viruses, as well as other inflammation-related diseases.

Thus, a selective inhibitor of mPGES-1 may serve as a therapeutic approach for treating and protecting particularly-vulnerable populations (older, male, and obese people), from severe COVID-19 disease progression and death. Furthermore, a selective inhibitor of mPGES-1 may serve as an improved therapeutic approach for treatment of other inflammation-related diseases, such as all types of acute and chronic pain, arthritis, stroke, sepsis, pneumonia, airway inflammation, heart failure, typhoid fever, and vascular inflammation such as abdominal aortic aneurysms (AAAs). However, development of a new drug is usually a long process; and, to date, no mPGES-1 inhibitor has been approved by the FDA.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method of inhibiting expression or activity of prostaglandin E2 (PGE₂) in a cell that involves contacting the cell with or introducing into the cell an effective amount of an mPGES-1 inhibitor, selected from the group consisting of ceftriaxone, aztreonam, cefotetan, pharmaceutically-acceptable salts thereof, and combinations thereof, wherein the contacting or introducing results in inhibition of expression or activity of PGE₂ in the cell. In some embodiments, the inhibitor is ceftriaxone or a pharmaceutically-acceptable salt thereof. In some embodiments, the inhibitor is aztreonam or a pharmaceutically-acceptable salt thereof. In some embodiments, the inhibitor is cefotetan or a pharmaceutically-acceptable salt thereof. In some embodiments, the inhibitor is provided in a pharmaceutical composition further comprising a pharmaceutically-acceptable carrier. In some embodiments, the cell is in a subject.

The presently-disclosed subject matter includes a method of treating coronavirus in a subject that involves administering to the subject an effective amount of an mPGES-1 inhibitor, selected from the group consisting of ceftriaxone, aztreonam, cefotetan, pharmaceutically-acceptable salts thereof, and combinations thereof. In some embodiments, the coronavirus is SARS-CoV-2. In some embodiments, the method also involves identifying the subject as having been exposed to the coronavirus, having tested positive for the coronavirus, and/or displaying one or more symptoms associated with the coronavirus. In some embodiments, the mPGES-1 inhibitor is administered without any additional active agents. In some embodiments, the method also involves administering an anti-viral agent. In some embodiments, the anti-viral agent is selected from the group consisting of remdesivir, chloroquine, hydroxychloroquine, oseltamivir, favipiravir, umifenovir, and galidesivir.

The presently-disclosed subject matter includes a method of reducing inflammation in a subject that involves administering to the subject an effective amount of an mPGES-1 inhibitor, selected from the group consisting of ceftriaxone, aztreonam, cefotetan, pharmaceutically-acceptable salts thereof, and combinations thereof. In some embodiments, the method also involves identifying the subject as having edema. In some embodiments, the method also involves identifying the subject as having stroke, sepsis, pneumonia, airway inflammation, heart failure, typhoid fever, or vascular inflammation. In some embodiments, the subject has vascular inflammation that is an abdominal aortic aneurysm (AAA).

The presently-disclosed subject matter includes a method of reducing pain in a subject that involves administering to the subject an effective amount of an mPGES-1 inhibitor, selected from the group consisting of ceftriaxone, aztreonam, cefotetan, pharmaceutically-acceptable salts thereof, and combinations thereof. In some embodiments, the method also involves identifying the subject as having pain. In some embodiments, the method also involves identifying the subject as having hyperalgesia. In some embodiments, the method also involves identifying the subject has having arthritis pain.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1C: illustrate dose-dependent inhibition of ceftriaxone (FIG. 1A), aztreonam (FIG. 1B), and cefotetan (FIG. 1C) against human mPGES-1.

FIGS. 2A and 2B illustrate the anti-inflammatory and analgesic effects of ceftriaxone (200 mg/kg, IP) on carrageenan-induced hyperalgesia (FIG. 2A) and carrageenan-induced paw edema (FIG. 2B) in wild-type rats (n=10 for each group) in comparison with a strong opioid drug (oxycodone, 5 mg/kg, IP). The hyperalgesia is represented by the Paw Withdrawal Latency (PWL). The paw edema was represented by the percent increase in the paw volume. Treatment (ceftriaxone or oxycodone or vehicle) was injected (IP) 1 h before 1% carrageenan (100 μL) injection.

FIGS. 3A and 3B illustrate the anti-inflammatory and analgesic effects of cefotetan (100 mg/kg, IP) on carrageenan-induced hyperalgesia (A) and carrageenan-induced paw edema (B) in wild-type rats (n=10 for each group) in comparison with a strong opioid drug (oxycodone, 5 mg/kg, IP). The hyperalgesia is represented by the Paw Withdrawal Latency (PWL). The paw edema was represented by the percent increase in the paw volume. Treatment (cefotetan or oxycodone or vehicle) was injected (IP) 1 h before 1% carrageenan (100 μL) injection.

FIG. 4 includes results of post-treatment of carrageenan-induced hyperalgesia in wild-type rats with ceftriaxone (100 or 200 mg/kg, IP) or oxycodone (5 mg/kg, IP) in comparison with the positive control (carrageenan and vehicle) and negative control (untreated).

FIG. 5 includes results of post-treatment of carrageenan-induced hyperalgesia in wild-type rats with cefotetan (50 or 100 mg/kg, IP) or oxycodone (5 mg/kg, IP) in comparison with the positive control (carrageenan and vehicle) and negative control (untreated).

FIG. 6 illustrates the anti-inflammatory and analgesic effects (in terms of arthritis score) of ceftriaxone and cefotetan on CFA-induced knee joint arthritis in wild-type rats (n=10 for each group). CFA was injected to the knee joint on Day 0. Drug or vehicle (control) was injected (IP) daily on Days 3 to 6. Arthritis score (mean spontaneous pain rating) measured on Days 0 to 6, 8, 10, and 12.

FIG. 7 illustrates the anti-inflammatory effects of ceftriaxone in terms of maximum aortic diameter.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is based in part on the discovery that ceftriaxone, aztreonam, and cefotetan have utility as microsomal prostaglandin E2 (PGE₂) synthase-1 (mPGES-1) inhibitors. Ceftriaxone, aztreonam, and cefotetan are FDA-approved injectable drugs, which heretofore have not been identified or suggested for use as mPGES-1 inhibitors.

Ceftriaxone is known for use as an antibiotic to treat infections such as gonorrhea and meningitis.

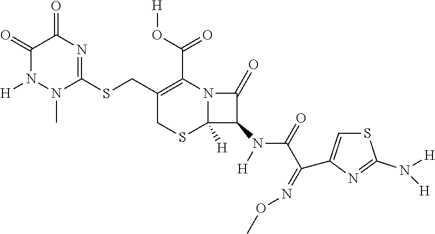 [SMILES: CO/N=C(\C(=O)N[C@@H]1C(=O)N2C(C(=O)O)=C(CSc3nc(=O)c(=O)[nH]n3C)CS[C@H]12)c1csc(N)n1]

Cefotetan is known as a cephamycin-type antibiotic.

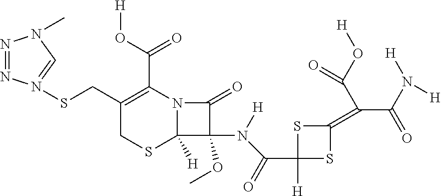

Aztreonam has been identified as an antibiotic useful for treating infections caused by gram-negative bacteria.

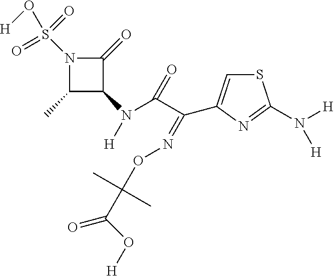 [SMILES: C[C@H]1[C@H](NC(=O)/C(=N\OC(C)(C)C(=O)O)c2csc(N)n2)C(=O)N1S(=O)(=O)O]

The presently-disclosed subject matter applies this unexpected and beneficial discovery, providing a method of inhibiting expression or activity of prostaglandin E2 (PGE2) in a cell. The presently-disclosed subject matter further includes a method of treating coronavirus in a subject, a method of reducing inflammation in a subject, and a method of reducing pain in a subject. As potent mPGES-1 inhibitors, ceftriaxone, aztreonam, and cefotetan are also contemplated to be effective for treatment a number of other inflammation-related diseases, including but not limited to various types of inflammatory pain and neuropathic pain, lupus, and skin disorders such as psoriasis and actinic keratosis. Such mPGES-1 inhibitors are contemplated to be more effective and safe for treatment of inflammation as compared to, for example, nonsteroidal anti-inflammatory drugs (NSAIDs) such as COX-1 and COX-2 inhibitors.

The presently-disclosed subject matter includes a method of inhibiting expression or activity of prostaglandin E2 (PGE2) in a cell, which involves contacting the cell with or introducing into the cell an effective amount of an mPGES-1 inhibitor, selected from the group consisting of ceftriaxone, aztreonam, cefotetan, pharmaceutically-acceptable salts thereof, and combinations thereof, wherein the contacting or introducing results in inhibition of expression or activity of PGE2 in the cell. In some embodiments, the cell is in a subject.

In some embodiments of the method of inhibiting PGE2, the inhibitor is ceftriaxone or a pharmaceutically-acceptable salt thereof. In some embodiments, the inhibitor is aztreonam or a pharmaceutically-acceptable salt thereof. In some embodiments, the inhibitor is cefotetan or a pharmaceutically-acceptable salt thereof.

The presently disclosed subject matter also includes a method of treating coronavirus, including protecting against or reducing the risk of a coronavirus infection and/or treating a coronavirus infection or a coronavirus disease, which involves administering to the subject an effective amount of an mPGES-1 inhibitor, selected from the group consisting of ceftriaxone, aztreonam, cefotetan, pharmaceutically-acceptable salts thereof, and combinations thereof. The coronavirus can be, for example, SARS-CoV or SARS-CoV-2.

In some embodiment, the method of treating coronavirus also involves identifying the subject as having been exposed to the coronavirus. In some embodiment, the method of treating coronavirus also involves identifying the subject as having tested positive for the coronavirus. In some embodiment, the method of treating coronavirus also involves identifying the subject as displaying one or more symptoms associated with the coronavirus.

In some embodiments the method also involves administering an anti-viral agent. In some embodiments the mPGES-1 inhibitor and the anti-viral agent are co-administered. In some embodiments, the anti-viral agent is selected from the group consisting of remdesivir, chloroquine, hydroxychloroquine, oseltamivir, favipiravir, umifenovir, and galidesivir.

The presently disclosed subject matter also includes a method of reducing inflammation in a subject, which involves administering to the subject an effective amount of an mPGES-1 inhibitor, selected from the group consisting of ceftriaxone, aztreonam, cefotetan, pharmaceutically-acceptable salts thereof, and combinations thereof.

In some embodiments, the method of reducing inflammation also involves identifying the subject as having a hyperinflammatory response or cytokine storm. In some embodiments, the method of reducing inflammation also involves identifying the subject as having edema. In some embodiments, the method of reducing inflammation also involves identifying the subject as having stroke, sepsis, pneumonia, airway inflammation, heart failure, typhoid fever, or vascular inflammation. In some embodiments, the subject has vascular inflammation that is an abdominal aortic aneurysm (AAA). In some embodiments, the subject has an inflammation-related disease.

In some embodiments, the inflammation-related disease is related to a bacteria, a virus, or another microbe. Examples of diseases related to a bacteria include Actinomycosis, Bacterial pneumonia, Brucellosis, Bubonic plague, Buruli ulcer, Campylobacteriosis, Cat-scratch disease, Chancroid, Chlamydia, Clostridium Difficile Infection, Diphtheria, Ehrlichiosis, Epidemic typhus, Erysipelas, Glanders, Granuloma inguinale, Group A streptococcal infection, Impetigo, Lemierre's syndrome, Legionellosis (Legionnaires Disease), Leprosy, Leptospirosis, Listeriosis, Lyme disease, Melioidosis, Meningitis, Meningococcal disease. Necrotizing fasciitis, Osteomyelitis, Paratyphoid fever, Plague, Pneumonic plague, Psittacosis, Q fever, Rat-bite fever, Relapsing fever, Rheumatic fever, Rocky Mountain spotted fever, Salmonellosis, Scarlet fever, Sepsis, Shigellosis, Staphylococcal scalded skin syndrome, Syphilis, Tetanus, Tularemia, Typhoid fever, Vibriosis (Vibrio), Whooping cough, and Yersiniosis.

Examples of diseases related to a virus or other microbe include Babesiosis, Chikungunya Virus Infection (Chikungunya), Dengue, 1,2,3,4 (Dengue Fever), Encephalitis, Enterovirus Infection, Granuloma inguinale, Haemophilus Influenza disease, Type B (Hib or H-flu), Hantavirus Pulmonary Syndrome (HPS), Hepatitis A (Hep A), Hepatitis B (Hep B), Hepatitis C (Hep C), Hepatitis D (Hep D), Hepatitis E (Hep E), Herpes, Histoplasmosis infection (Histoplasmosis), Human Papillomavirus (HPV), Influenza (Flu), Malaria, Measles, Meningitis, Viral (Meningitis, viral), Middle East Respiratory Syndrome Coronavirus (MERS-CoV), Mumps, Norovirus, Powassan, Rubella, Scabies, Severe Acute Respiratory Syndrome (SARS), Varicella (Chickenpox), West Nile Virus, and Yellow Fever, and Zika Virus Infection (Zika).

In some embodiments, the inflammation-related disease is an autoimmune disease.

Examples of autoimmune diseases include Ankylosing spondylitis, Anti-GBM/Anti-TBM nephritis, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Chagas disease, Chronic inflammatory demyelinating polyneuropathy (CIDP), Crohn's disease, Dermatomyositis, Discoid lupus, Endometriosis, Eosinophilic esophagitis (EoE), Eosinophilic fasciitis, Fibromyalgia, Fibrosing alveolitis, Giant cell arteritis (temporal arteritis), Giant cell myocarditis, Glomerulonephritis, Granulomatosis with Polyangiitis, Herpes gestationis or pemphigoid gestationis (PG), Inclusion body myositis (IBM), Juvenile arthritis, Juvenile diabetes (Type 1 diabetes), Juvenile myositis (JM), Kawasaki disease, Leukocytoclastic vasculitis, Lupus, Microscopic polyangiitis (MPA), Myositis, Neonatal Lupus, Palindromic rheumatism (PR), Pars planitis (peripheral uveitis), Perivenous encephalomyelitis, Polymyalgia rheumatic, Polymyositis, Primary sclerosing cholangitis, Progesterone dermatitis, Psoriasis, Psoriatic arthritis, Reactive Arthritis, Relapsing polychondritis, Rheumatic fever, Rheumatoid arthritis, Sarcoidosis, Scleritis, Subacute bacterial endocarditis (SBE), Takayasu's arteritis, Temporal arteritis/Giant cell arteritis, Transverse myelitis, Type 1 diabetes, Ulcerative colitis (UC), Uveitis, and Vasculitis.

In some embodiments of the methods disclosed herein, the mPGES-1 inhibitor is administered without any additional active agent.

In some embodiments of the methods disclosed herein, the mPGES-1 inhibitor is provided in a pharmaceutical composition further comprising a pharmaceutically-acceptable carrier.

In some embodiments of the methods disclosed herein, the compound that is contacted, introduced, and/or administered can be provided in a in a pharmaceutical composition further comprising a pharmaceutically-acceptable carrier. As used herein, the term “pharmaceutically acceptable carrier” refers to sterile aqueous or nonaqueous solutions, dispersions, suspensions or emulsions, as well as sterile powders for reconstitution into sterile solutions or dispersions just prior to use.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In some embodiments, administration of a known compound can made by known routes for administering that compound. For example, in some embodiments, if ceftriaxone is administered, it can be administered by injection, which is a known route for administration of ceftriaxone.

As used herein, the terms “inhibit”, “inhibitor”, or “inhibiting” are not meant to require complete inhibition, but refers to a reduction in target activity. Such reduction can be a reduction of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

As used herein, an “activity” of a polypeptide, such as an enzyme, refers to any activity exhibited by the polypeptide, such as catalyzing a particular biochemical reaction. Such activities can be empirically determined using methods known to those of ordinary skill in the art.

As used herein, “expression” refers to the process by which polypeptides are produced by transcription and translation of polynucleotides. The level of expression of a polypeptide can be assessed using any method known in art.

As used herein, the term “subject” refers to a human or animal subject. In some embodiments, the subject is a mammal. In some aspects, subject is a rodent. In other aspects of the invention, subject is a human.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: FDA-Approved Drugs Identified as mPGES-1 Inhibitors

The screening protocol (DREAM-in-CDM) used to identify the mPGES-1 inhibitors as described herein consists of three steps: structure-based virtual screening of FDA-approved drugs; in vitro activity assays; and clinical data mining. The virtual screening was performed by using a multiple-step computational screening procedure described previously (Zhou et al., 2017) and the protein conformation of human mPGES-1 (Zhou et al., 2019), predicting that ceftriaxone, aztreonam, and cefotetan are inhibitors of human mPGES-1. Ceftriaxone, aztreonam, and cefotetan that all belong to antibiotic injectables, and were not previously known or suggested for use in connection with mPGE-1 inhibition.

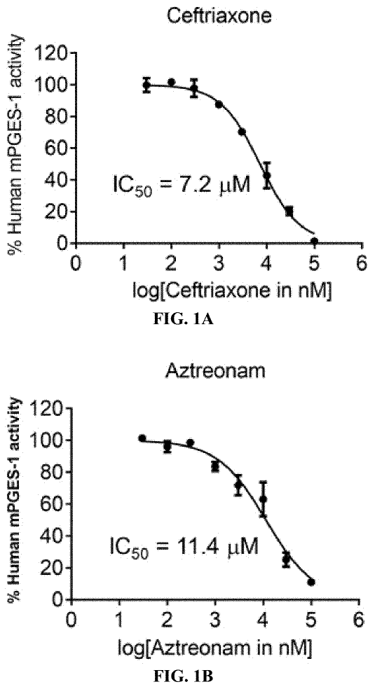

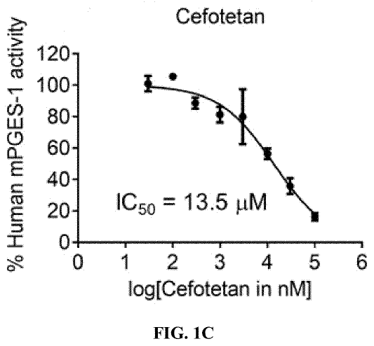

The inhibitory activity of these drugs against human mPGES-1 was determined in vitro using the previously described in vitro activity procedure, an enzyme-linked immunosorbent assay (ELISA). (Ding et al., 2018a; Ding et al., 2018b; Zhou et al., 2017). According to the obtained in vitro activity data (FIGS. 1A-1C), these drugs can inhibit human mPGES-1 with IC₅₀ (the concentration for inhibiting mPGES-1 by 50%) being 7.2, 11.4, and 13.5 μM for ceftriaxone, aztreonam, and cefotetan, respectively.

### Example 2: Clinically Effective Concentrations

As the efficacy of a drug is dependent on both the IC₅₀ and the actual drug concentration achieved in the body. In clinical data mining, it was first considered whether any of these drugs can reach possibly effective in vivo concentrations in human plasma that should be higher than the corresponding IC₅₀ values.

To address this question, the pharmacokinetic (PK) data was collected, particularly the maximum drug concentrations (Cₘₐₓ) associated with the specific human doses used, along with the FDA-approved maximum doses. As seen in Table 1, the FDA-approved maximum doses of these drugs are all very high (implying the limited side effects): 4 g per day for both ceftriaxone and cefotetan or 2 g per day for aztreonam.

TABLE 1
IC₅₀ (μM) and Cₘₐₓ (μM) of drugs identified herein as mPGES-1
inhibitors in human plasma from clinical pharmacokinetic data.

| | | | | Cₘₐₓ | |
|---|---|---|---|---|---|
| Drug (ref. | | | | in human | Maximum dose |
| for the | IC₅₀ | Dose used in | | plasma | approved |
| clinical data) | (μM) | clinical trial | | (μM) | by FDA |
| Ceftriaxone | 7.2 ± 2.4 | 2 | g, IV | 463 | 4 g |
| (Patel et al., | | | | | |
| 1981) | | | | | |
| Aztreonam | 11.4 ± 1.1 | 500 | mg, IV | 129 | 2 g |
| (Swabb et al., | | | | | |
| 1983) | | | | | |
| Cefotetan | 13.5 ± 4.0 | 1 | g, IV | 440 | 4 g |
| (Nakagawa et | | | | | |
| al., 1982) | | | | | |

Each drug, with a dose lower than the maximum dose allowed by the FDA, can still reach a Cₘₐₓ much higher than the corresponding IC₅₀ against mPGES-1, suggesting that all these drugs may serve as effective anti-inflammatory drugs within the FDA-approved dose ranges. In particular, ceftriaxone at a dose of 2 g (a half of the maximum dose allowed by the FDA) has Cₘₐₓ=463 μM which is ˜64-fold higher than its IC₅₀ (7.2 μM) against mPGES-1. Hence, ceftriaxone may be used as a highly effective anti-inflammatory drug.

### Example 3: Analysis of Preclinical and Clinical Data

All available preclinical and clinical data relevant to ceftriaxone were thoroughly collected and analyzed.

Through virtual screening, followed by in vitro enzyme activity assays and clinical data mining, ceftriaxone, aztreonam, and cefotetan were been identified as potent mPGES-1 inhibitors, which can be repurposed to as anti-inflammatory drugs to treat, for example, COVID-19.

Interestingly, there have been plenty of animal studies demonstrating anti-inflammatory and analgesic effects of ceftriaxone.(Chen et al., 2012; Hu et al., 2010; Yimer et al., 2019) According to the used animal models of inflammation and pain/hyperalgesia,(Chen et al., 2012; Hu et al., 2010; Yimer et al., 2019) a number of proinflammatory mediators were upregulated and glutamate transporter 1 (GLT-1) was downregulated in certain models. Administration of ceftriaxone effectively attenuated the proinflammatory cytokines (including TNF-α) and reversed the downregulation of GLT-1. Thus, the authors of these studies attributed the observed anti-inflammatory and analgesic effects of ceftriaxone to its ability to reverse the downregulation of GLT-1, without knowing any specific human protein target of ceftriaxone for these favorable effects.

All these effects of ceftriaxone are consistent with the mPGES-1 inhibition by ceftriaxone. The correlation between elevated PGE₂ levels and elevated proinflammatory cytokines is known, as described hereinabove. Regarding GLT-1, it is known that the elevated PGE₂ levels negatively correlate with the downregulated glutamate transporter levels and positively correlate with the increased extracellular glutamate levels, and that administration of an inhibitor of COX-2 or mPGES-1 was able to lower the elevated PGE₂ and glutamate levels and increase the downregulated glutamate transporter levels.(Chen et al., 2013; Soldner et al., 2019)

Being consistent with the anti-inflammatory and analgesic effects in animal models, previous placebo-controlled, double-blind study (Caperton et al., 1990) demonstrated that ceftriaxone was efficacious in treatment of chronic inflammatory arthritis. However, without knowing any host protein target of ceftriaxone, the authors (Caperton et al., 1990) speculated that the “patients may have an occult bacterial infection underlying their chronic inflammatory arthritis, and may respond to antibiotic therapy.”

With ceftriaxone identified herein as an mPGES-1 inhibitor, it can be better understood that the previously observed broad anti-inflammatory and analgesic effects in both preclinical and clinical studies were most likely due to its effective inhibition of mPGES-1. By potently inhibiting mPGES-1, ceftriaxone may be capable of blocking/attenuating the hyperinflammatory response and enhancing host immune response against the viral infection, according to the roles of selective mPGES-1 inhibition (Smeitink et al., 2020) discussed above. Thus, ceftriaxone may serve as a potentially effective treatment to prevent the patients from severe COVID-19 disease progression and death.

Further, according to a report on clinical characteristics of 138 hospitalized COVID-19 patients in Wuhan, China, ceftriaxone has been used as one of the antibacterial therapy options: moxifloxacin, 89 [64.4%]; ceftriaxone, 34 [24.6%]; azithromycin, 25 [18.1%].(Wang et al., 2020a). But the authors did not attempt to collect data concerning the efficacy of any specific drug.

With the knowledge as disclosed herein that ceftriaxone can also effectively inhibit mPGES-1 as an anti-inflammatory drug, in addition to its well-known antibacterial activities, the present inventors propose that ceftriaxone can be used more often in the future as this drug alone can serve as both anti-inflammatory and antibacterial treatments.

Azithromycin, an antibiotic that is commonly sold as Zithromax Z-Pak® (Pfizer), has been used together with hydroxychloroquine (antiviral treatment) in ongoing clinical trials for treatment of COVID-19 patients.(Pfizer, 2020) Additionally, it is contemplated to provide a combination of ceftriaxone with an antiviral drug (e.g. remdesivir or hydroxychloroquine) as an efficacious in treatment of COVID patients.

Since remdesivir has been approved by the FDA for emergency use to treat COVID-19 patients, it is contemplated that a combined use of ceftriaxone and remdesivir (or another effective antiviral drug) may be a useful option to combat the COVID, although ceftriaxone can be used alone for treatment of COVID.

In addition, as an inhibitor of mPGES-1, ceftriaxone etc. may also be repurposed to treat a number of other inflammation-related diseases, such as various forms of pain, cardiovascular diseases, neurodegenerative diseases, and various types of cancer.(Hanaka et al., 2009; Koeberle and Werz, 2015; Radmark and Samuelsson, 2010)

### Example 4: Cytoprotective Activity of Ceftriaxone Against SARS-CoV-2 Infection

Ceftriaxone was tested to determine its cytoprotective activity against SARS-CoV-2 infection through cell culture. It was found that ceftriaxone was indeed cytoprotective against SARS-CoV-2 infection (data not shown).

### Example 5: Anti-Inflammatory and Analgesic Effects of Ceftriaxone or Cefotetan Pre-Treatment on Carrageenan-Induced Paw Edema and Carrageenan-Induced Hyperalgesia

To examine the anti-inflammatory and analgesic effects of the drugs, they were tested against carrageenan-induced paw edema and hyperalgesia in comparison with oxycodone, a well-known strong opioid drug. For oxycodone tablets, the FDA-approved maximum dose for the first-time users is 40 mg; single doses higher than 40 mg are only for use in opioid-tolerant patients. The rat dose corresponding to an human equivalent dose (HED) of 40 mg (for an average human body weight of 60 kg) is (40/60)×6.3=4.2 mg/kg, according to the generally accepted animal-human dose conversion guide. (Nair et al., 2016). Thus, 5 mg/kg oxycodone was used in the tests. For ceftriaxone or cefotetan, a rat dose of 200 mg/kg corresponds to a HED of 2 g, and a rat dose of 100 mg/kg corresponds to a HED of 1 g.

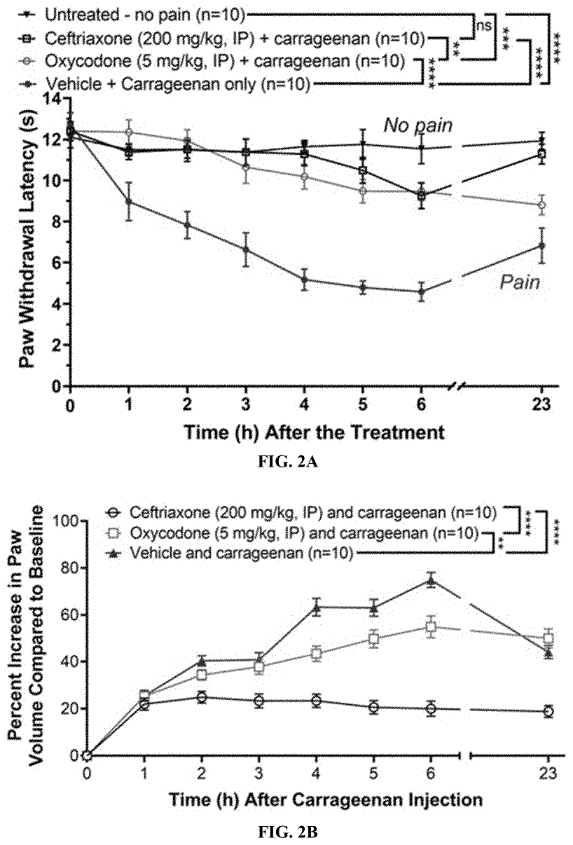

In the carrageenan-induced hyperalgesia model, the hyperalgesia/pain is reflected by the paw withdrawal latency (PWL). The shorter the PWL time, the more severe the pain. As shown in FIG. 2A, pre-treatment with 200 mg/kg ceftriaxone completely suppressed carrageenan-induced hyperalgesia. There was no significant difference in PWL between the ceftriaxone treatment group and the control group (untreated rats without pain at all). In comparison, oxycodone also effectively relieved the pain within six hours after oxycodone injection. However, there was still significant pain at 23 h (the next day). So, 200 mg/kg ceftriaxone was even more effective than 5 mg/kg oxycodone in pain relief.

In the carrageenan-induced paw edema model, the paw edema is reflected by the percent increase in the paw volume. As seen in FIG. 2B, ceftriaxone significantly decreased the percent increase in the paw volume. So, ceftriaxone showed significant anti-inflammatory effect. In comparison, oxycodone slowed down the percent increase in the paw volume but, eventually, there was no significant difference between the oxycodone treatment group and the control group (with carrageenan only) at 23 h (next day) in the percent increase in the paw volume.

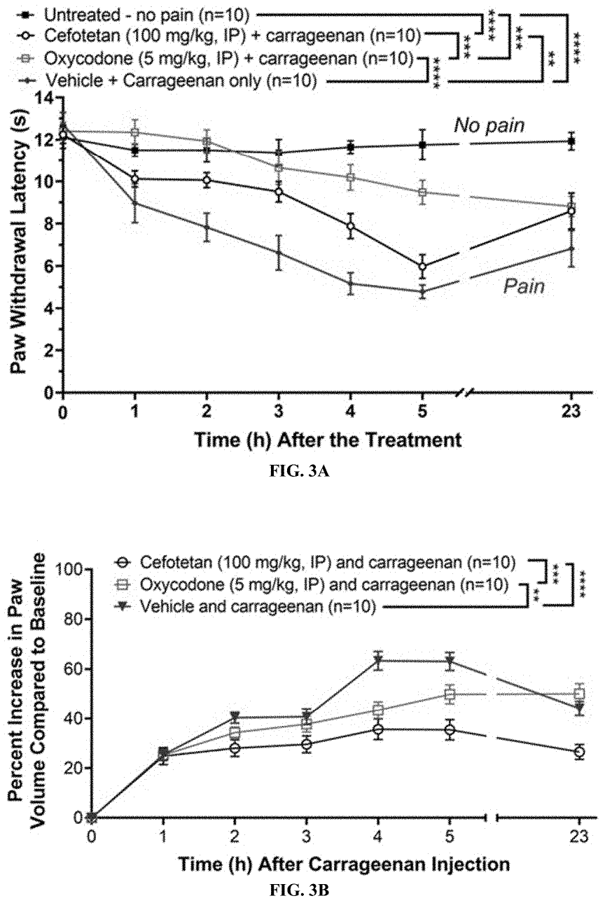

Similarly, cefotetan also has the desired anti-inflammatory and analgesic effects as shown in FIGS. 3A and 3B.

### Example 6: Analgesic Effects of the Post-Treatment on Carrageenan-Induced Hyperalgesia

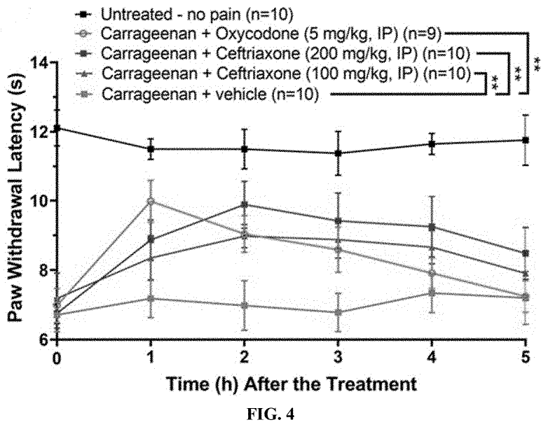

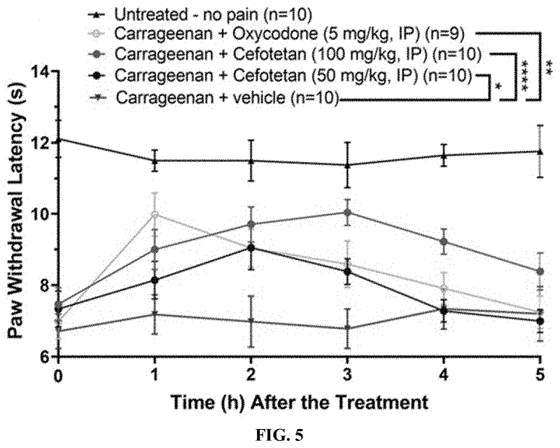

To better model the clinical treatment of pain, ceftriaxone (FIG. 4) and cefotetan (FIG. 5) were also tested in a post-treatment model of carrageenan-induced hyperalgesia. Rats were first injected with carrageenan to induce the hyperalgesia/pain. In the next day (23 h later), there was still persistent pain (see FIGS. 4 and 5 for vehicle control group—bottom line), allowing the post-treatment effects of the drugs to be tested in comparison with oxycodone.

At 23 h after the carrageenan injection, the rats were treated with ceftriaxone, cefotetan, oxycodone, or vehicle (control). As shown in FIGS. 4 and 5, administration of 5 mg/kg oxycodone significantly relieved the pain, as expected. The PWL peak time for oxycodone was 1 h. Ceftriaxone or cefotetan also significantly, and dose-dependently, relieved the pain.

### Example 7: Anti-Inflammatory Effects on Adjuvant-Induced Knee Joint Arthritis

Ceftriaxone and cefotetan were further tested using an adjuvant-induced knee joint arthritis model described by Hammell et al. 2016. Using this one-week knee joint arthritis model, CFA was injected to the knee joint on Day 0 to induce chronic arthritis and associated pain, followed by daily PWL and arthritis score (mean spontaneous pain rating) assessments from Day 0 to Day 7.

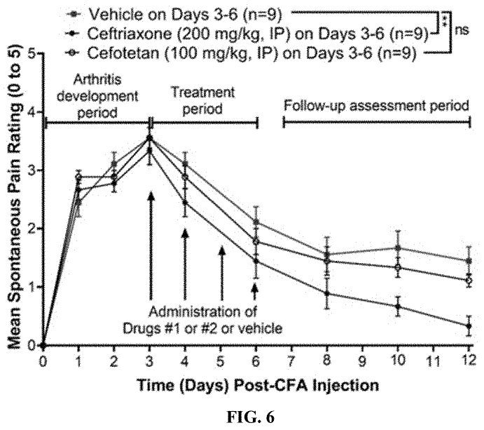

Daily treatment with ceftriaxone and cefotetan or vehicle (control group) started on Day 3 (after the arthritis score assessment on that day) and continued until Day 6. There were a total of four doses of the respective treatment from Day 3 to Day 6. After the CFA injection on Day 0, the arthritis score rapidly increased (FIG. 6) beginning Day 1. On Day 3, the arthritis score reached the peak (reflecting the most severe arthritis), which is consistent with previous observations reported for the control group. (Hammell et al., 2016). During the assessment period, the mean spontaneous pain rating was reduced in association with celriaxone or cefotetan treatment.

### Example 8: Effectiveness in Treatment of Abdominal Aortic Aneurysm (AAA)

Ceftriaxone was also tested in treatment of angiotensin (AngII)-induced AAA model. This experiment was performed using ApoE-deficient mice. AngII (with a minipump to continuously release the AngII solution within 28 days) was used to induce AAA in this strain of mice beginning Day 0 for all animals in both control and intervention groups.

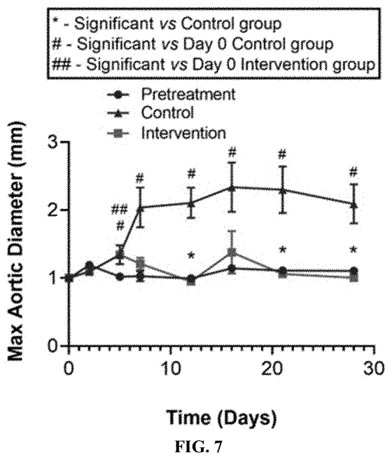

The AAA was characterized by the maximum aortic diameter of the vein detected by the ultrasound method. Ceftriaxone was given beginning on Day 5 for intervention. As shown in FIG. 7, ceftriaxone intervention significantly stopped the progression of AAA, and it even reversed the AAA.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Aliabadi, F.; Ajami, M.; Pazoki-Toroudi, H., Why does COVID-19 pathology have several clinical forms? BioEssays 2020, 42, 2000198. doi.org/10.1002/bies.202000198.
Apostolova, E.; Lukova, P.; Baldzhieva, A.; Katsarov, P.; Nikolova, M.; Iliev, I.; Peychev, L.; Trica, B.; Oancea, F.; Delattre, C.; Kokova, V, Immunomodulatory and Anti-Inflammatory Effects of Fucoidan: A Review. Polymers (Basel) 2020, 12, 2338. doi: 10.3390/polym12102338.
Bergqvist F., Morgenstern R., Jakobsson P-J., A review on mPGES-1 inhibitors: From preclinical studies to clinical applications, Prostaglandins & Other Lipid Mediators 2019, 147, 106383, ISSN 1098-8823, doi.org/10.1016/j.prostaglandins.2019.106383.
Blanco-Melo, D.; Nilsson-Payant, B. E.; Liu, W.-C.; Uhl, S.; Hoagland, D.; Møller, R.; Jordan, T. X.; Oishi, K.; Panis, M.; Sachs, D.; Wang, T. T.; Schwartz, R. E.; Lim, J. K.; Albrecht, R. A.; tenOever, B. R., Imbalanced Host Response to SARS-CoV-2 Drives Development of COVID-19. Cell 2020, 181, 1036-1045
Bormann, T.; Maus, R.; Stolper, J.; Jonigk, D.; Welte, T.; Gauldie, J.; Kolb, M. R.; Maus, U. A., Role of the COX2-PGE 2 axis in S. pneumoniae induced exacerbation of experimental fibrosis. Am. J. Physiol. Lung Cell Mol. Physiol. 2020, Dec. 9. doi: 10.1152/ajplung.00024.2020.
Caperton, E. M., Heim-Duthoy, K. L., Matzke, G. R., Peterson, P. K., and Johnson, R. C. (1990). Ceftriaxone Therapy of Chronic Inflammatory Arthritis. Arch Intern Med 150, 1677-1682. doi:1610.1001/archinte.1990.00040031677015.
Chen, R., Zhang, J., Fan, N., Teng, Z.-q., Wu, Y, Yang, H., Tang, Y-p., Sun, H., Song, Y, and Chen, C. (2013). D9-THC-Caused Synaptic and Memory Impairments Are Mediated through COX-2 Signaling. Cell 155, 1154-1165.
Chen, Z., He, Y, and Wang, Z. J. (2012). The beta-lactam antibiotic, ceftriaxone, inhibits the development of opioid-induced hyperalgesia in mice. Neuroscience letters 509, 69-71.
Cheng, Y, Wang, M., Yu, Y, Lawson, J., Funk, C. D., and Fitzgerald, G. A. (2006a). Cyclooxygenases, microsomal prostaglandin E synthase-1, and cardiovascular function. J Clin Invest 116, 1391-1399.
Cheng, Y, Wang, M., Yu, Y, Lawson, J., Funk, C. D., and FitzGerald, G. A. (2006b). Cyclooxygenases, microsomal prostaglandin E synthase-1, and cardiovascular function. J Clin Invest 116, 1391-1399.
Chini, M. G., Giordano, A., Potenza, M., Terracciano, S., Fischer, K., Vaccaro, M. C., Colarusso, E., Bruno I., Riccio, R., Koeberle, A., Werz, O., Bifolco G. (2020) Targeting mRGES-1 by Combinatorial Approach, Identification of the Amiobenzothiazole Scaffold to Suppress PGE₂ Levels. ACS Medicinal Chemistry Letters, 11, 783-789.
Chua, F., Armstrong-James, D., Desai, S. R., Barnett, J., Kouranos, V, Kon, O. M., Jose, R., Vancheeswaran, R., Loebinger, M. R., Wong, J., et al. (2020). The role of CT in case ascertainment and management of COVID-19 pneumonia in the UK: insights from high-incidence regions. Lancet Respir Med March 25, pii: S2213-2600(20)30132-6. doi: 10.1016 S2213-2600(20)30132-6. [Epub ahead of print].
Ding, K., Zhou, Z., Hou, S., Yuan, Y, Zhou, S., Zheng, X., Chen, J., Loftin, C., Zheng, F., and Zhan, C.-G. (2018a). Structure-based discovery of mPGES-1 inhibitors suitable for preclinical testing in wild-type mice as a new generation of anti-inflammatory drugs. Scientific Reports 8, 5205. doi:5210.1038/s41598-41018-23482-41594.
Ding, K., Zhou, Z., Zhou, S., Yuan, Y, Kim, K., Zhang, T., Zheng, X., Zheng, F., and Zhan, C.-G. (2018b). Design, synthesis, and discovery of 5-((1,3-diphenyl-1H-pyrazol-4-yl)methylene)pyrimidine-2,4,6(1H,3H,5H)-triones and related derivatives as novel inhibitors of mPGES-1. Bioorg Med Chem Letters 28, 858-862.
Engblom, D., Saha, S., Engstrom, L., Westman, M., Audoly, L. P., Jakobsson, P. J., and Blomqvist, A. (2003). Microsomal prostaglandin E synthase-1 is the central switch during immune-induced pyresis. Nature Neurosci 6, 1137-1138.
Gomez, I.; Foudi, N.; Longrois, D.; Norel, X., The role of prostaglandin E2 in human vascular inflammation. Prostaglandins Leukot Essent Fatty Acids 2013, 89, 55-63.
Gross, S., Tilly, P., Hentsch, D., Vonesch, J. L., and Fabre, J. E. (2007). Vascular wall-produced prostaglandin E2 exacerbates arterial thrombosis and atherothrombosis through platelet EP3 receptors. J Exp Med 204, 311-320.
Hammell, D. C.; Zhang, L. P.; Ma, F.; Abshire, S. M.; McIlwrath, S. L.; Stinchcomb, A. L.; Westlund, K. N., Transdermal cannabidiol reduces inflammation and pain-related behaviours in a rat model of arthritis. Eur J Pain 2016, 20 (6), 936-948.
Hanaka, H., Pawelzik, S.-C., Johnsen, J. I., Rakonjac, M., Terawaki, K., Rasmuson, A., Sveinbjornsson, B., Schumacher, M. C., Hamberg, M., Samuelsson, B., et al. (2009). Microsomal prostaglandin E synthase 1 determines tumor growth in vivo of prostate and lung cancer cells. Proc Natl Acad Sci USA 106, 18757-18762.
Hong, W.; Chen, Y; You, K.; Tan, S.; Wu, F.; Tao, J.; Chen, X.; Zhang, J.; Xiong, Y; Yuan, F.; Yang, Z.; Chen, T.; Chen, X.; Peng, P.; Tai, Q.; Wang, J.; Zhang, F.; Li, Y-X., Celebrex Adjuvant Therapy on Coronavirus Disease 2019: An Experimental Study. Front. Pharmacol. 2020, 11, 561674. doi: 10.3389/fphar.2020.561674.
Hu, Y, Li, W., Lu, L., Cai, J., Xian, X., Zhang, M., Li, Q., and Li, L. (2010). An anti-nociceptive role for ceftriaxone in chronic neuropathic pain in rats. Pain 148, 284-301.
Ibuprofen activities (www.selleckchem.com/products/Ibuprofen (Advil).html; last seen: Apr. 22, 2020).
Insuela, D. B. R.; Ferrero, M. R.; Coutinho, D. d. S.; Martins, M. A.; Carvalho, V. F., Could Arachidonic Acid-Derived Pro-Resolving Mediators Be a New Therapeutic Strategy for Asthma Therapy? Front. Immunol. 2020, 11, 580598. doi: 10.3389/fimmu.2020.580598.
Kaithwas, G.; Mukherjee, A.; Chaurasia, A. K.; Majumdar, D. K., Anti-inflammatory, analgesic and antipyretic activities of Linum usitatissimum L. (flaxseed/linseed) fixed oil. Indian J. Exp. Biol. 2011, 49(12), 932-938.
Koeberle, A., and Werz, O. (2015). Perspective of microsomal prostaglandin E2 synthase-1 as drug target in inflammation-related disorders. Biochem Pharmacol 98, 1-15.
Kojima, F., Kapoor, M., Yang, L., Fleishaker, E. L., Ward, M. R., Monrad, S. U., Kottangada, P. C., Pace, C. Q., Clark, J. A., Woodward, J. G., et al. (2008). Defective generation of a humoral immune response is associated with a reduced incidence and severity of collagen-induced arthritis in microsomal prostaglandin E synthase-1 null mice. J Immunol 180, 8361-8368.
Kojima, F., Matnani, R. G., Kawai, S., Ushikubi, F., and Crofford, L. J. (2011). Potential roles of microsomal prostaglandin E synthase-1 in rheumatoid arthritis. Inflamm Regen 31, 157-166.
Kox, M.; B. Waalders, N. J.; Kooistra, E. J.; Gerretsen, J.; Pickkers, P., Cytokine Levels in Critically Ill Patients With COVID-19 and Other Conditions. JAMA 2020, 324(15), 1565-1567. doi: 10.1001/jama.2020.17052.Kudo, I., and Murakami, M. (2005). Prostaglandin E synthase, a terminal enzyme for prostaglandin E-2 biosynthesis. J Biochem Mol Biol 38, 633-638.
Kupferschmidt, K., and Cohen, J. (2020). WHO launches global megatrial of the four most promising coronavirus treatments, Science News on Mar. 22, 2020. Posted in: HealthCoronavirus. doi:10.1126/science.abb8497.
Li, L.; Yu, Y; Hou, R.; Hao, J.; Jiang, J., Inhibiting the PGE 2 Receptor EP2 Mitigates Excitotoxicity and Ischemic Injury. ACS Pharmacol. Transl. Sci. 2020, 3(4), 635-643. doi: 10.1021/acsptsci.0c00040.
Ma, W.; Li, L.; Xing, S., PGE2/EP4 receptor and TRPV1 channel are involved in repeated restraint stress-induced prolongation of sensitization pain evoked by subsequent PGE2 challenge. Brain Res. 2019, 1721, 146335. doi: 10.1016/j.brainres.2019.146335.
Mehta, P., McAuley, D. F., Brown, M., Sanchez, E., Tattersall, R. S., and Manson, J. J. (2020). COVID-19: consider cytokine storm syndromes and immunosuppression. Lancet 395, 1033-1034.
Minhas, P. S.; Latif-Hernandez, A.; McReynolds, M. R.; Durairaj, A. S.; Wang, Q.; Rubin, A.; Joshi6, A. U.; He, J. Q.; Gauba, E.; Liu, L.; Wang, C.; Linde, M.; Sugiura, Y; Moon, P. K.; Majeti, R.; Suematsu, M.; Mochly-Rosen, D.; Weissman, I. L.; Longo, F. M.; Rabinowitz, J. D.; Andreasson, K. I., Restoring metabolism of myeloid cells reverses cognitive decline in ageing. Nature 2021, Published online: 20 Jan. 2021 (doi.org/10.1038/s41586-020-03160-0).
Nair, A. B.; Jacob, S., A simple practice guide for dose conversion between animals and human. J. Basic Clin. Pharm. 2016, 7, 27-31.
Nakagawa, K., Koyama, M., Tachibana, A., Komiya, M., Kikuchi, Y, and Yano, K. (1982). Pharmacokinetics of cefotetan (YM09330) in humans. Antimicrobial agents and chemotherapy 22, 935-941.
NPR (2020). Antiviral Drug Remdesivir Shows Promise For Treating Coronavirus In NIH Study (www.npr.org/sections/health-shots/2020/04/29/848034963/antiviral-drug-remdesivir-shows-promise-for-treating-coronavirus-in-nih-study).
Pace, S., Rossi, A., Krauth, V., Dehm, F., Troisi, F., Bilancia, R., Weinigel, C., Rummler, S., Werz, O., and Sautebin, L. (2020). Sex differences in prostaglandin biosynthesis in neutrophils during acute inflammation. Sci Rep 7(1), 3759. doi: 3710.1038/s41598-41017-03696-41598.
Patel, I. H., Chen, S., Parsonnet, M., Hackman, M. R., Brooks, M. A., Konikoff, J., and Kaplan, S. A. (1981). Pharmacokinetics of ceftriaxone in humans. Antimicrob Agents Chemother 20, 634-641.
Pfizer reports safety data of azithromycin in Covid-19 trial (www.clinicaltrialsarena.com/news/pfizer-data-azithromycin-covid-19-trial/), 26 Mar. 2020.
Radmark, O., and Samuelsson, B. (2010). Microsomal prostaglandin E synthase-1 and 5-lipoxygenase: potential drug targets in cancer. J Intern Med 268, 5-14.
Reis, M. B.; Rodrigues, F. L.; Lautherbach, N.; Kanashiro, A.; Sorgi, C. A.; Meirelles, A. F. G.; Silva, C. A. A.; Zoccal, K. F.; Souza, C. O. S.; Ramos, S. G.; Matsuno, A. K.; Rocha, L. B.; Salgado, H. C.; Navegantes, L. C. C.; Kettelhut, Í. C.; Cupo, P.; Gardinassi, L. G.; Faccioli, L. H., Interleukin-1 receptor-induced PGE 2 production controls acetylcholine-mediated cardiac dysfunction and mortality during scorpion envenomation. Nat. Commun. 2020, 11(1), 5433. doi: 10.1038/s41467-020-19232-8.
Rodriguez-Morales, A. J., Cardona-Ospina, J. A., Gutiérrez-Ocampo, E., Villamizar-Peña, R., Holguin-Rivera, Y, Escalera-Antezana, J. P., Alvarado-Arnez, L. E., Bonilla-Aldana, D. K., Franco-Paredes, C., Henao-Martinez, A. F., et al. (2020). Clinical, laboratory and imaging features of COVID-19: A systematic review and meta-analysis. Travel Med Infect Dis, March 13:101623. doi: 101610.101016/j.tmaid.102020.101623. [Epub ahead of print].
Saha, S., Engström, L., Mackerlova, L., Jakobsson, P. J., and Blomqvist, A. (2005). Impaired febrile responses to immune challenge in mice deficient in microsomal prostaglandin E synthase-1. Am J Physiol Regul Integr Comp Physiol 288, 100-107.
Sampey, A. V., Monrad, S., and Crofford, L. J. (2005). Microsomal prostaglandin E synthase-1: the inducible synthase for prostaglandin E2. Arthritis Res Ther 7, 114-117.
Sander, W. J., O'Neill, H. G., and Pohl, C. H. (2017). Prostaglandin E2 As a Modulator of Viral Infections. Front Physiol 8, 89.
Schoenberger, S. D., Kim, S. J., Sheng, J., Rezaei, K. A., Lalezary, M., and Cherney, E. (2012). Increased Prostaglandin E2 (PGE2) Levels in Proliferative Diabetic Retinopathy, and Correlation with VEGF and Inflammatory Cytokines. Invest Ophthalmol Vis Sci 53, 5906-5911.
Serhan, C. N., and Levy, B. (2003). Success of prostaglandin E-2 in structure-function is a challenge for structure-based therapeutics. Proc Natl Acad Sci USA 100, 8609-8611.
Smeitink, J.; Jiang, X.; Pecheritsyna, S.; Renkema, H.; Maanen, R. v.; Beyrath, J., Hypothesis: mPGES-1-derived Prostaglandin E2, a so far missing link in COVID-19 pathophysiology?Preprints, www.preprints.org/manuscript/202004.0180/v1. 2020.
Soldner, E. L. B., Hartz, A. M. S., Akanuma, S.-I., Pekcec, A., Doods, H., Kryscio, R. J., Hosoya, K.-I., and Bauer, B. (2019). Inhibition of human microsomal PGE2 synthase-1 reduces seizure-induced increases of P-glycoprotein expression and activity at the blood-brain barrier. FASEB Journal 33, 13966-13981.
St-Jacques, B., and Ma, W. (2011). Role of prostaglandin E2 in the synthesis of the proinflammatory cytokine interleukin-6 in primary sensory neurons: an in vivo and in vitro study. J Neurochem 118, 841-854.
Stebbing, J., Phelan, A., Griffin, I., Tucker, C., Oechsle, O., Smith, D., and Richardson, P. (2020). COVID-19: combining antiviral and anti-inflammatory treatments. Lancet 20, 400-401.
Stebbing, J.; Krishnan, V.; Bono, S. d.; Ottaviani, S.; Casalini, G.; Richardson, P. J.; Monteil, V.; Lauschke, V. M.; Mirazimi, A.; Youhanna, S.; Tan, Y-J.; Baldanti, F.; Sarasini, A.; Terres, J. A. R.; Nickoloff, B. J.; Higgs, R. E.; Rocha, G.; Byers, N. L.; Schlichting, D. E.; Nirula, A.; Cardoso, A.; Corbellino, M.; Sacco-Baricitinib-Study-Group, Mechanism of baricitinib supports artificial intelligence-predicted testing in COVID-19 patients. EMBO Mol. Med. 2020b, 12(8), e12697. doi: 10.15252/emmm.202012697.
Sungnak, W., Huang, N., Bécavin, C., and Berg, M. (2020). SARS-CoV-2 Entry Genes Are Most Highly Expressed in Nasal Goblet and Ciliated Cells within Human Airways. Available from: arxiv.org/abs/2003.06122.
Swabb, E. A., Singhvi, S. M., Leitz, M. A., Frantz, M., and Sugerman, A. (1983). Metabolism and pharmacokinetics of aztreonam in healthy subjects. Antimicrob Agents Chemother 24, 394-400.
Tanioka, T., Nakatani, Y, Semmyo, N., Murakami, M., and Kudo, I. (2000). Molecular identification of cytosolic prostaglandin E2 synthase that is functionally coupled with cyclooxygenase-1 in immediate prostaglandin E2 biosynthesis. J Biol Chem 275, 32775-32782.
Trebino, C. E., Stock, J. L., Gibbons, C. P., Naiman, B. M., Wachtmann, T. S., Umland, J. P., Pandher, K., Lapointe, J. M., Saha, S., Roach, M. L., et al. (2003). Impaired inflammatory and pain responses in mice lacking an inducible prostaglandin E synthase. Proc Natl Acad Sci USA 100, 9044-9049.
Vieira, A. S.; Araldi, D.; Dias, E. V.; Prado, F. C. d.; Tambeli, C. H.; Parada, C. A., Janus kinase 2 activation participates in prostaglandin E2-induced hyperalgesia. Life Sci. 2016, 166, 8-12.
Wang, D., Hu, B., Hu, C., Zhu, F., Liu, X., Zhang, J., Wang, B., Xiang, H., Cheng, Z., Xiong, Y, et al. (2020a). Clinical Characteristics of 138 Hospitalized Patients With 2019 Novel Coronavirus-Infected Pneumonia in Wuhan, China. JAMA 323, 1061-1069. doi:1010.1001/jama.2020.1585.
Wang, M., Cao, R., Zhang, L., Yang, X., Liu, J., Xu, M., Shi, Z., Hu, Z., Zhong, W., and Xiao, G. (2020b). Remdesivir and chloroquine effectively inhibit the recently emerged novel coronavirus (2019-nCoV) in vitro. Cell Research 30, 269-271; doi.org/210.1038/s41422-41020-40282-41420.
Wang, M., Lee, E., Song, W., Ricciotti, E., Rader, D. J., Lawson, J. A., Puré, E., and FitzGerald, G. A. (2008). Microsomal prostaglandin E synthase-1 deletion suppresses oxidative stress and angiotensin II-induced abdominal aortic aneurysm formation. Circulation 117, 1302-1309.
Wang, M., Zukas, A. M., Hui, Y, Ricciotti, E., Puré, E., and FitzGerald, G. A. (2006). Deletion of microsomal prostaglandin E synthase-1 augments prostacyclin and retards atherogenesis. Proc Natl Acad Sci USA 103, 14507-14512.
Yimer, E. M., Hishe, H. Z., and Tuem, K. B. (2019). Repurposing of the β-Lactam Antibiotic, Ceftriaxone for Neurological Disorders: A Review. Front Neurosci, 26 Mar. 2019 doiorg/103389/fnins201900236 13, 236. doi.org/210.3389/fnins.2019.00236.
Zhang, W., Zhao, Y, Zhang, F., Wang, Q., Li, T., Liu, Z., Wang, J., Qin, Y, Zhang, X., Yan, X., et al. (2020). The use of anti-inflammatory drugs in the treatment of people with severe coronavirus disease 2019 (COVID-19): The experience of clinical immunologists from China. Clinical Immunology 214, 108393.
Zhou, P., Yang, X.-L., Wang, X.-G., Hu, B., Zhang, L., Zhang, W., Si, H.-R., Zhu, Y, Li, B., Huang, C.-L., et al. (2020a). A pneumonia outbreak associated with a new coronavirus of probable bat origin. Nature 579, 270-273.
Zhou, S., Zhou, Z., Ding, K., Yuan, Y, Loftin, C., Zheng, F., and Zhan, C.-G. (2020b). DREAM-in-CDM Approach and Identification of a New Generation of Anti-inflammatory Drug Targeting mPGES-1. Sci Rep, in press.
Zhou, S., Zhou, Z., Ding, K., Yuan, Y, Zheng, F., and Zhan, C.-G. (2019). In Silico Observation of the Conformational Opening of the Glutathione-binding Site of Microsomal Prostaglandin E2 Synthase-1. J Chem Inf Modeling 59, 3839-3845.
Zhou, Z., Yuan, Y, Zhou, S., Ding, K., Zheng, F., and Zhan, C.-G. (2017). Selective inhibitors of human mPGES-1 from structure-based computational screening. Biorg Med Chem Letters 27, 3739-3743.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of inhibiting expression or activity of prostaglandin E2 (PGE₂) in a cell, comprising contacting the cell with or introducing into the cell an effective amount of an mPGES-1 inhibitor, selected from the group consisting of ceftriaxone, aztreonam, cefotetan, pharmaceutically-acceptable salts thereof, and combinations thereof, wherein the contacting or introducing results in inhibition of expression or activity of PGE₂ in the cell.

2. The method of claim 1, wherein the inhibitor is ceftriaxone or a pharmaceutically-acceptable salt thereof.

3. The method of claim 1, wherein the inhibitor is aztreonam or a pharmaceutically-acceptable salt thereof.

4. The method of claim 1, wherein the inhibitor is cefotetan or a pharmaceutically-acceptable salt thereof.

5. The method of claim 1, wherein the inhibitor is provided in a pharmaceutical composition further comprising a pharmaceutically-acceptable carrier.

6. The method of claim 1, wherein the cell is in a subject.

7. A method of treating coronavirus in a subject, comprising administering to the subject an effective amount of an mPGES-1 inhibitor, selected from the group consisting of ceftriaxone, aztreonam, cefotetan, pharmaceutically-acceptable salts thereof, and combinations thereof.

8. The method of claim 7, wherein the coronavirus is SARS-CoV-2.

9. The method of claim 7, and further comprising identifying the subject as having been exposed to the coronavirus, having tested positive for the coronavirus, and/or displaying one or more symptoms associated with the coronavirus.

10. The method of claim 7, wherein the mPGES-1 inhibitor is administered without any additional active agents.

11. The method of claim 7, and further comprising administering an anti-viral agent.

12. The method of claim 11, wherein the anti-viral agent is selected from the group consisting of remdesivir, chloroquine, hydroxychloroquine, oseltamivir, favipiravir, umifenovir, and galidesivir.

13. A method of reducing inflammation in a subject, comprising administering to the subject an effective amount of an mPGES-1 inhibitor, selected from the group consisting of ceftriaxone, aztreonam, cefotetan, pharmaceutically-acceptable salts thereof, and combinations thereof.

14. The method of claim 13, and further comprising identifying the subject as having edema.

15. The method of claim 13, and further comprising identifying the subject as having stroke, sepsis, pneumonia, airway inflammation, heart failure, typhoid fever, or vascular inflammation.

16. The method of claim 15, wherein the subject has vascular inflammation that is an abdominal aortic aneurysm (AAA).

17. A method of reducing pain in a subject, comprising administering to the subject an effective amount of an mPGES-1 inhibitor, selected from the group consisting of ceftriaxone, aztreonam, cefotetan, pharmaceutically-acceptable salts thereof, and combinations thereof.

18. The method of claim 17, and further comprising identifying the subject as having pain.

19. The method of claim 17, and further comprising identifying the subject as having hyperalgesia.

20. The method of claim 17, and further comprising identifying the subject has having arthritis pain.
